(19) United States
(12) Patent Application Publication
(10) Pub. No.: US 2006/0199832 A1
(43) Pub. Date: Sep. 7, 2006

(54) Use of non-immunosupressive pipecolic acid derivatives for inducing chondrogenic differentiation

(75) Inventors: Fusako Nishigaki, Tokyo (JP); Susumu Miyata, Tokyo (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: Astellas Pharma Inc., Tokyo (JP)

(21) Appl. No.: 10/547,782

(22) PCT Filed: Mar. 1, 2004
(86) PCT No.: PCT/JP04/02502

§ 371 Date: Sep. 6, 2005

(30) Foreign Application Priority Data
Mar. 4, 2003 (AU) 2003901023

(51) Int. Cl. A61K 31/4745

(52) U.S. Cl. 514/291

(57) ABSTRACT

A non-immunosuppressive pipecolic acid derivative having an affinity for FKBP-type immunophilins is provided for inducing chondrogenic differentiation. Composition containing such compounds is also disclosed.

### BACKGROUND ART

Certain immunosuppressive pipecolic acid derivative having an affinity for FKBP-type immunophiline, such as FK506, are known to induce chondrogenic differentiation (WO00/74665).

WO96/40140 shows that certain non-immunosuppressive pipecolic acid derivative, having an affinity for FKBP-type immunophiline, has an activity for stimulating growth of damaged peripheral nerves or for promoting neuronal regeneration.

The compound (1) per se mentioned below, is known (WO89/05304). And it is also known to have neuroprotective efficacy (WO01/05385) and to have a high level of neurotrophic activity and low level of immunosuppressive activity (WO02/053159).

However, non-immunosuppressive pipecolic acid derivative having an affinity for FKBP-type immunophiline has never been known to induce chondrogenic differentiation.

### DISCLOSURE OF INVENTION

The inventors of this invention have found that the non-immunosuppressive pipecolic acid derivative having an affinity for FKBP-type immunophiline has an inducing activity of chondrogenic differentiation.

Accordingly, this invention provides a new use of the non-immunosuppressive pipecolic acid derivative having an affinity for FKBP-type immunophilins for inducing chondrogenic differentiation.

Further, this invention provides an agent for inducing chondrogenic differentiation, which comprises the non-immunosuppressive pipecolic acid derivative having an affinity for FKBP-type immunophilins.

Still further, this invention provides a method for inducing chondrogenic differentiation, which comprises administering said non-immunosuppressive pipecolic acid derivative having an affinity for FKBP-type immunophilins to mammals.

Still further, this invention provides a method for preventing or treating damages of cartilage, which comprises administering said non-immunosuppressive pipecolic acid derivative having an affinity for FKBP-type immunophilins to mammals.

The preferable “FKBP-type immunophilins” of “non-immunosuppressive pipecolic acid derivative having an affinity for FKBP-type immunophilins” in the present invention is FKBP-12. The binding affinity thereof can be evaluated by well-known binding assay methods. For example, “Binding Assay to FKBP12” that was shown in WO02/053159, Example 5, can be exemplified.

The immunosuppressive activity of “non-immunosuppressive pipecolic acid derivative” can be evaluated by well-known test methods. For example, “Inhibition of IL-2 Production of Tricyclic Compounds (I)” (TEST 12 of U.S. Pat. No. 4,929,611) or “Mixed lymphocyte Reaction (MLR)” (WO02/053159, Example 5) can be exemplified therefor. The “non-immunosuppressive pipecolic acid derivative” in the present invention means ‘pipecolid acid derivatives’ which do not have immunosuppressive activity, substantially. More particularly, it does not inhibit a production of IL-2, substantially.

As a preferable example of “non-immunosuppressive pipecolic acid derivative having an affinity for FKBP-type immunophilins”, the compounds that were shown in WO 96/40140 can be exemplified, the disclosures of which are incorporated herein by reference.

The most preferable “non-immunosuppressive pipecolic acid derivative having an affinity for FKBP-type immunophilins” is a compound (1) of the following compound.

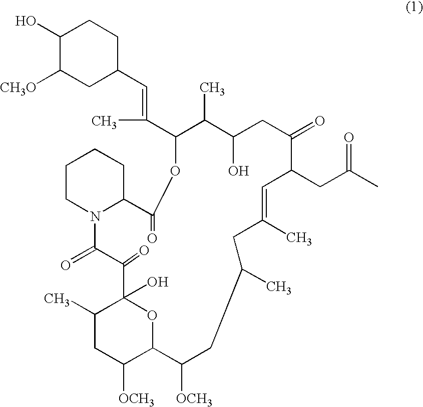

The compound (1) maybe in a form of its salt, which includes conventional non-toxic and pharmaceutically acceptable salt such as the salt with inorganic or organic bases, specifically, an alkali metal salt such as sodium salt and potassium salt, an alkali earth metal salt such as calcium salt and magnesium salt, an ammonium salt and an amine salt such as triethylamine salt and N-benzyl-N-methylamine salt.

With respect to the compound (1) used in the present invention, it is to be understood that there may be conformers and one or more stereoisomers such as optical and geometrical isomers due to asymmetric carbon atom(s) or double bond(s), and such conformers and isomers are also included within the scope of compound (1) in the present invention. And further, the compound (1) can be in the form of a solvate, which is included within the scope of the present invention. The solvate preferably include a hydrate and an ethanolate.

The “non-immunosuppressive pipecolic acid derivative having an affinity for FKBP-type immunophilins” usable in the present invention may be administered as pure compounds or mixtures of compounds or preferably, in a pharmaceutical vehicle or carrier.

The pharmaceutical compositions of this invention can be used in the form of a pharmaceutical preparation, for example, in solid, semisolid or liquid form, which contains the macrolide compounds of the present invention, as an active ingredient, in admixture with an organic or inorganic carrier or excipient suitable for external(topical), enteral, intravenous, intramuscular, or parenteral applications. The active ingredient may be compounded, for example, with the usual non-toxic, pharmaceutically acceptable, carriers for tablets, pellets, capsules, eye drops, suppositories, solutions (saline, for example), emulsion, suspensions (olive oil, for example), ointment and any other form suitable for use. The carriers which can be used are water, glucose, lactose, gum acacia, gelatin, mannitol, starch paste, magnesium trisilicate, talc, corn starch, keratin, colloidal silica, potato starch, urea and other carriers suitable for use in manufacturing preparations, in solid, semisolid, or liquid form, and in addition auxiliary, stabilizing, thickening and coloring agents and perfumes may be used. The active object compound is included in the pharmaceutical composition in an effective amount sufficient to produce the desired effect upon the process or condition of the disease.

Mammals which may be treated using the method of the present invention include livestock mammals such as cows, horses, etc., domestic animals such as dogs, cats, rats, etc. and humans.

While the dosage of therapeutically effective amount of the “non-immunosuppressive pipecolic acid derivative having an affinity for FKBP-type immunophilins” varies from and also depends upon the age and condition of each individual patient to be treated, a daily dose of about 0.0001-1000 mg, preferably 0.001-500 mg and more preferably 0.01-100 mg of the active ingredient is generally given for treating diseases, and an average single dose of about 0.001-0.01 mg, 0.2-0.5 mg, 1 mg, 5 mg, 10 mg, 50 mg, 100 mg, 250 mg and 500 mg is generally administered. Daily doses for chronic administration in humans will be in the range of about 0.1-0.3 mg/kg/day.

The following examples illustrate the present invention in further detail, it being to be understood that those examples are not intended to limit the scope of the invention.

### EXAMPLE 1

The inducing activity by the compound (1) on chondrogenic differentiation was evaluated in accordance with the below-mentioned method.

(1) The ATDC5 cell line provided by RIKEN Cell Bank (Tsukuba, Japan) was grown in a 1:1 mixture of Dulbecco's modified Eagle's medium and Ham's F12 medium (Nikken Biomedical Laboratory, Kyoto, Japan) supplemented with 10% heat-inactivated fetal bovine serum (Intergen, Purchase, N.Y.). Under these conditions, ATDC5 cells remain chondroprogenitor-like and do not express cartilage phenotypes.

(2) The above ATDC5 cells were plated in 12-multiwell plastic plate at a density of 1×10⁵ cells/well in the medium. After 4 hr, the medium was replaced with fresh medium containing the compound (1) or Cyclosporin A (CsA), and the culture was continued for a 24 days with medium change every 2 or 3 days. Cells were fixed with methanol and stained 0.1% Alcian blue (Sigma Chemical Co., St. Louis, Mo.) dissolved in 0.1 M hydrochloric acid for 16 hr at room temperature. Cells were then rinsed three times with distilled water, and the amount of cell-associated dye was measured at 620 nm after extraction with 6 M guanidine-HCl (300 μl/well)

Results

ATDC5 cells were incubated with the compound (1) or CsA for 24 days and the amount of proteoglycan was assayed.

The compound (1) induced differentiation into chondrocyte in a concentration-dependent manner (1-10000 ng/ml). On the other hand, CsA did not induce the differentiation. The results were shown in FIG. 1.

The above results indicate that the “non-immunosuppressive pipecolic acid derivative having an affinity for FKBP-type immunophilins”, such as the compound (1), are useful for preventing or treating damages of cartilage (e.g., hyaline cartilage, fibrocartilage, elastic cartilage) which are caused by external injury, inflammatory diseases, autoimmune diseases, and so on.

More particularly, the present agent is useful for preventing or treating failure of chondrocyte, such as chondrodystrophy, arthritis (e.g., rheumatoid arthritis, osteoarthritis, etc); osteoporosis; and so on.

And further, the macrolides of the present invention is also useful for regeneration of tissues, such as connective tissue (e.g., cartilaginous tissue) and/or bone tissue.

The patents, patent applications and publications cited herein are incorporated by reference.

### BRIEF DESCRIPTION OF THE DRAWINGS

FIG. 1 shows the effect of the compound (1) on chondrogenic differentiation of ATDC5 cells.

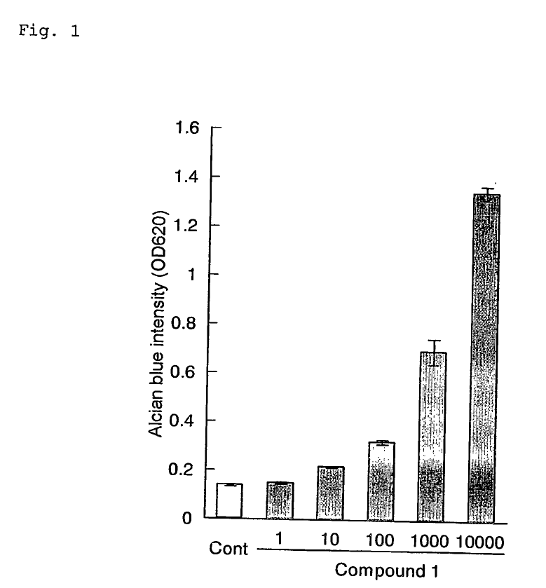

1. A method for inducing chondrogenic differentiation, which comprises administering an effective amount of a non-immunosuppressive pipecolic acid derivative having an affinity for FKBP-type immunophilins to mammals to prevent or treat damages of cartilage.

2. The method of claim 1, wherein the non-immunosuppressive pipecolic acid derivative having an affinity for FKBP-type immunophilins is a compound (1) of the following compound.

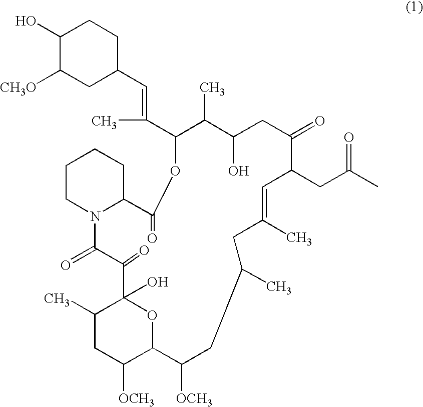

3. The method of claim 1, wherein the damages of cartilage is a failure of chondrocyte or osteoporosis.

4. The method of claim 1, wherein the damages of cartilage is hyaline cartilage, fibrocartilage, elastic cartilage, chondrodystrophy, rheumatoid arthritis, osteoarthritis, or osteoporosis.

5. A use of a non-immunosuppressive pipecolic acid derivative having an affinity for FKBP-type immunophilins for manufacturing a medicament for preventing or treating damages of cartilage.

6. A pharmaceutical composition for inducing chondrogenic differentiation, which comprises a non-immunosuppressive pipecolic acid derivative having an affinity for FKBP-type immunophilins in admixture with a carrier or excipient.

7. A commercial package comprising the pharmaceutical composition containing non-immunosuppressive pipecolic acid derivative having an affinity for FKBP-type immunophilins and a written matter associated therewith, wherein the written matter states that said non-immunosuppressive pipecolic acid derivative having an affinity for FKBP-type immunophilins can or should be used for preventing or treating damages of cartilage.
